(19) United States
(12) Patent Application Publication
(10) Pub. No.: US 2007/0093764 A1
(43) Pub. Date: Apr. 26, 2007

(54) Infusion device for administering fluids to a patient

(75) Inventor: Ramon Guerrero, Plano, TX (US)

Correspondence Address: MICHAEL E. MARTIN;GARDERE WYNNE SEWELL LLP, 3000 THANKSGIVING TOWER, 1601 ELM STREET, SUITE 3000, DALLAS, TX, 75201, US

(73) Assignee: INTRASAFE MEDICAL. LLC, Frisco, TX (US)

(21) Appl. No.: 11/286,880

(22) Filed: Nov. 23, 2005

Related U.S. Application Data
(63) Continuation-in-part of application No. 11/257,890, filed on Oct. 25, 2005.

(51) Int. Cl. A61M 25/00

(52) U.S. Cl. 604/284; 604/93.01; 248/200

(57) ABSTRACT

A fluid infusion device includes multiple angular oriented fluid inlet ports connected to a manifold via one way valves, and a further port allowing fluid flow to or from the manifold. Each of the inlet ports may have a Luer fitting and a self-sealing swabable valve adapted for receiving a syringe. A tubing connector is adapted for receiving a mating connector at an outlet end of the manifold. A mounting bracket on the manifold or on a housing of the device cooperates with typical hospital equipment support structure.

### CROSS REFERENCE TO RELATED APPLICATION

This application is a continuation-in-part of copending application Ser. No. 11/257,890, filed Oct. 25, 2005.

### TECHNICAL FIELD

The present invention generally relates to medical devices, particularly devices for anesthesiology and critical care, more particularly to medical devices used to administer multiple medicines and other agents to a patient, and to methods for more effectively administering multiple fluids to a patient.

### BACKGROUND

The intravenous (IV) administration of medicines by medical personnel, such as anesthetic agents by an anesthesiologist, is a complex procedure. IV medicine administration in the form of needlesticks poses serious risk for the healthcare practitioner. Additionally, unless carefully controlled, IV medicine administration poses a risk to the patient of nosocomial (hospital acquired) infections. For example, because multiple anesthesia medicines are to be administered closely after one another, such administration requires the careful and rapid infusion of a series of different drugs, such as a hypnotic agent, a muscle relaxant, and a narcotic.

This series of anesthetic agents has typically been administered by separately handling multiple syringes to sequentially transfer the medicines into an intravenous port, one at a time, preferably in rapid succession to minimize the patient's pain and, in some cases, to expedite the patient's drowsiness or unconsciousness. Consequently, an anesthesiologist administering these three agents typically must rapidly perform the following steps: (1) take the first syringe; (2) insert it into an intravenous catheter; (3) press down on the syringe to transfer the medicine into the intravenous catheter leading to a patient entry site; (4) remove the syringe; (5) place it somewhere in the patient's hospital room, such as on the patient's bed; then take the second syringe and repeat the steps 1 through 5; and, then take the third syringe and repeat steps 1 through 5.

The above described approach has a number of drawbacks. For example, it is uncommon for the healthcare practitioner to sterilize the injection port in between injections. This can potentially lead to admission of bacteria into the sterile IV system. It also does not allow the dosage to be easily controlled, as needed, from patient to patient. A syringe may become contaminated laying on the patient's bed or may actually be knocked to the floor, such as in an emergency operation; and the rapid insertion and removal of syringes with needles is problematic as the needles may accidentally stick the patient, doctor, or nurse, which is especially dangerous, as it dramatically increases the potential transmission of certain diseases or viruses. Moreover, since multiple syringes are needed to induce unconsciousness, the anesthesiologist's hands are unnecessarily used to hold syringes, which makes the anesthesiologist less efficient.

Various techniques, such as stopcocks and similar manifold systems, have been introduced to overcome these drawbacks but have fallen short of effectively protecting the healthcare practitioner and the patients. In order to interpose a stopcock or similar manifold , the practitioner must typically interrupt the fluid flow of an IV line, disconnect the tubing, interpose the manifold system and then reconnect the IV tubing. This lends itself to the introduction of bacteria into a patient's sterile IV line.

A significant advance in overcoming these drawbacks was realized by the invention and development of an infusion medical device described in U.S. Pat. No. 6,508,791, assigned to the assignee of the present invention. This multiple needleless injection port device, because of its unique design, among other advantages, enables the efficient and coordinated infusion of multiple drugs and other agents to the patient. It eliminates the risk of needlesticks and avoids a break in the IV fluid path, thus reducing the risk or danger of contamination or harm to either the patient or the medical personnel.

### SUMMARY

This invention is directed to new and unique improvements in a multiple port infusion device generally of the type described in U.S. Pat. No. 6,508,791. Specifically, the plural inlet ports are arranged in a specific angular pattern which uniquely improves the device's utility. In addition, a separate port is connected to the device's manifold for introducing or evacuating fluids from the manifold. A specially designed bracket is provided to enable convenient access and mounting of the device. Still further, the manifold, ports and connecting conduit sections are preferably formed of substantially rigid molded plastic, for example. These and other features and advantages of this invention will become readily apparent to one of ordinary skill in the art from the following description, taken in connection with the accompanying drawings, in which:

### BRIEF DESCRIPTION OF THE DRAWINGS

FIG. 1 is a front elevation view of an embodiment of an infusion device in accordance with the present invention, with the front side of the housing removed;

FIG. 2A is a top plan view of the infusion device shown in FIG. 1, with both the front and back sides of the housing in place;

FIG. 2B is a front perspective view of the infusion device shown in FIG. 1, with both the front and back sides of the housing in place;

FIG. 2C is a perspective view of the back side of the infusion device shown in FIG. 1 further illustrating a unique mounting bracket;

FIG. 3A is a front elevation view of another embodiment of the infusion device of the present invention with a front housing side removed;

FIG. 3B is a front perspective view of the infusion device shown in FIG. 3A;

FIG. 4 is a central section view of the infusion device in accordance with the invention;

FIG. 5 is a perspective view of a typical arrangement of an embodiment of the infusion device in accordance with the invention, connected to an injection site for an intravenous line shown in use to deliver fluids to a patient;

FIG. 6 is an elevation view of an embodiment of an infusion device, with both front and back housing sides removed, and an outlet conduit having a tubing connection for connecting to tubing leading to a patient entry point;

FIG. 7 is a view taken from the line 7-7 of FIG. 6;

FIG. 8 is a section view taken along line 8-8 of FIG. 7 showing a swabable self-sealing valve mounted to a Luer fitting and showing a check valve for the infusion device of the invention;

FIG. 9 is a view similar to FIG. 8 showing a syringe needle inserted in the swabable valve;

FIG. 10 is an elevation view of an embodiment of the infusion device of the present invention, with the front and back housing sides removed;

FIG. 11 is an elevation view of an embodiment of the infusion device of the present invention showing inlet tubing threadedly attached to each of the Luer fittings of the swabable valves;

FIG. 12 is an elevation view of an embodiment of the infusion device of the present invention showing a syringe attached to selected ones of self-sealing swabable valves;

FIG. 13 illustrates an embodiment of the infusion device of the present invention, showing the mounting bracket on the back side of the device housing, ready to be mounted to a cooperating receiving bracket;

FIG. 14 illustrates an embodiment of the infusion device of the present invention, showing the device mounting bracket being placed in the receiving bracket,

FIG. 15 illustrates an embodiment of the infusion device showing the device mounted on a receiving bracket,

FIG. 16 is a perspective view showing the device mounted on a flat-plate style receiving bracket; and

FIG. 17 is another perspective view showing the device mounted on the flat-plate style receiving bracket shown in FIG. 16.

### DETAILED DESCRIPTION OF THE PREFERRED EMBODIMENTS

In the following discussion, details are set forth to provide a thorough understanding of the present invention. However, those skilled in the art will appreciate that the present invention may be practiced without such specific details. Certain conventional and known elements have been illustrated in schematic form in order not to obscure the present invention in unnecessary detail. The subject matter of U.S. Pat. No. 6,508,791, issued Jan. 21, 2003 to Ramon Guerrero, is incorporated herein by reference. Several of the components of the infusion device disclosed and claimed may be formed of medical grade opaque or transparent substantially rigid plastic materials, for example. Accordingly, in the drawing figures, some of the components are drawn to show hidden lines of certain features indicating that the components may be formed as transparent. Like parts are marked throughout the specification and drawings with the same reference numerals.

The device of this invention includes a number of features, all of which work together to provide advantageous results. The detailed description herein of the preferred embodiments will lead to an understanding by those skilled in the art of its advantages to patients and healthcare personnel.

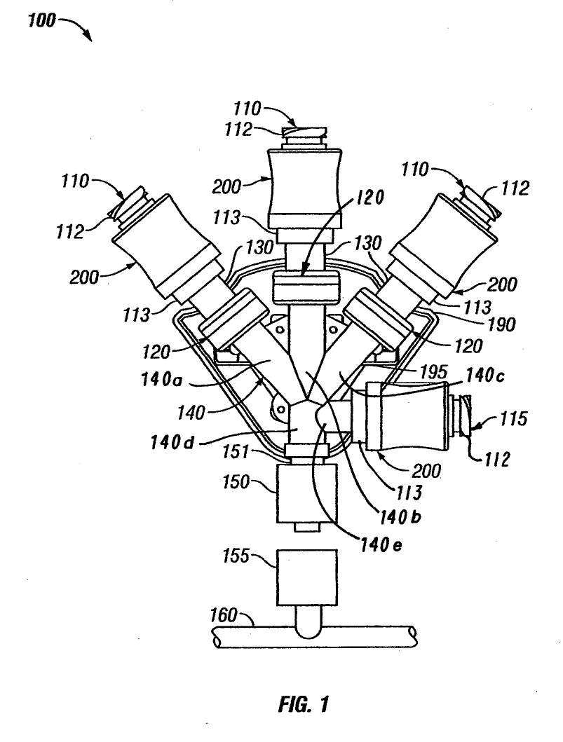

Referring now, to FIG. 1, an infusion device in accordance with the invention 100 comprises a plurality of generally upward facing inlet ports 110 including self-sealing receptacles comprising swabable valves 200 mounted to respective couplings, preferably Luer fittings 113, connected to respective one way or so-called check valves 120. A “swabable valve” is a valve whose entire surface that is exposed to the environment is capable of being wiped or swabbed with a disinfectant to eliminate bacteria or other contaminants. Swabable valves 200 and check valves 120 may be of types commercially available such as from Halkey-Roberts Corp., St. Petersburg, Fla., for example. Valves from other commercial sources may be used. Check valves 120 are mounted on a manifold 140 comprising respective conduits 140a, 140b and 140c disposed at, preferably, acute angles (more than 0° and less than 90°) with respect to each other and connected to a common outlet conduit 140d. A side inlet/outlet conduit 140e is connected to and extends at a right angle to conduit 140d, and conduit 140e is connected to a swabable valve 200 directly without a check valve interposed the valve 200 and the conduit 140d. Each self-sealing receptacle or valve 200 may include a Luer connector type helical cam or thread 112 adapted in a known way to be coupled to a syringe (not shown in FIG. 1) to provide for the flow of fluid from the syringe into and through the inlet port 110 to which it is attached. Self-sealing receptacles or swabable valves 200 can be cleaned and reused by swabbing with an antiseptic. Receptacles or valves 200 can also be replaced by conventional syringe needle receptive self-sealing elastomer valves, not shown. Still further, ports 110 may utilize other forms of closable valves or be connected to other fluid dispensing devices.

Each check valve 120 is operable to be in fluid flow communication with a respective port 110 to enable fluid to flow from the port 110 into and through manifold 140 but not in the opposite direction. Each port 110 is typically in fluid communication with an associated check valve 120 via a short conduit section 130, but the check valves 120 may be directly coupled to the ports 1 10, if desired. The self-sealing receptacles 200 may be permanently joined to the respective conduit sections 130 or directly to the one-way valves 120. As mentioned above, inlet port 115 is connected to manifold 140 via conduit 140e downstream in the direction of fluid flow through manifold 140 with respect to check valves 120. Port 115 may be an outlet port also for evacuating air trapped in manifold 140, for example. Port 115 includes a swabable valve 200 connected via a Luer connector 113 to manifold 140 and does not include a check valve interposed the valve 200 and the manifold. Additional ports similar to ports 110 and 115 may be provided, if desired, and oriented in different directions with respect to manifold 140. However, the orientation of the ports 110 is of importance with respect to ease of manipulation of syringes connected to the respective ports so that each individual syringe may be actuated and otherwise manipulated without interfering with any of the other syringes. Still further, the overall length of each of the conduit sections 140a, 140b, 140c and the connecting conduit sections 130 of each port 110 is kept to a minimum while allowing for the components of the structure to be provided, including the check valves 120 and the self-sealing receptacles or swabable valves 200. Still further, the internal diameters of the passageways formed by conduit sections 130, 140a, 140b, 140c, 140e and 140d are also minimized. Among other advantages this minimal length and diameter eliminates “dead space” in the channels, enabling enhanced control over the medication or other agent delivery. A preferred diameter of the passageways in each of these conduit sections is about 1.0 mm, for example. The manifold 140, conduit sections 130, valves 128, fittings 112 and 113 and the housings for swabable valves 200 are all formed of substantially rigid molded plastic and may be secured to each other by chemical or thermal bonding to form a rigid integrated structure.

The ports 110 are preferably adapted to be connected to either needle-less or needle-bearing syringes (not shown in FIG. 1). A conventional needle-less syringe has, for example, a stub end comprising a male Luer fitting that may fit into, and open, the swabable valve of each of the self-sealing receptacles 200, respectively, and may also have a connector part (not shown) that can be releasably connected to the Luer thread or cam 112 of self-sealing receptacle or swabable valve 200 to secure the syringe thereto. As shown in FIG. 11, the ports 110 can be modified to provide tubings 170 connected to them, respectively, by use of Luer type male connectors 171 coupled to the threads or cams 112 on the ends of the ports 110.

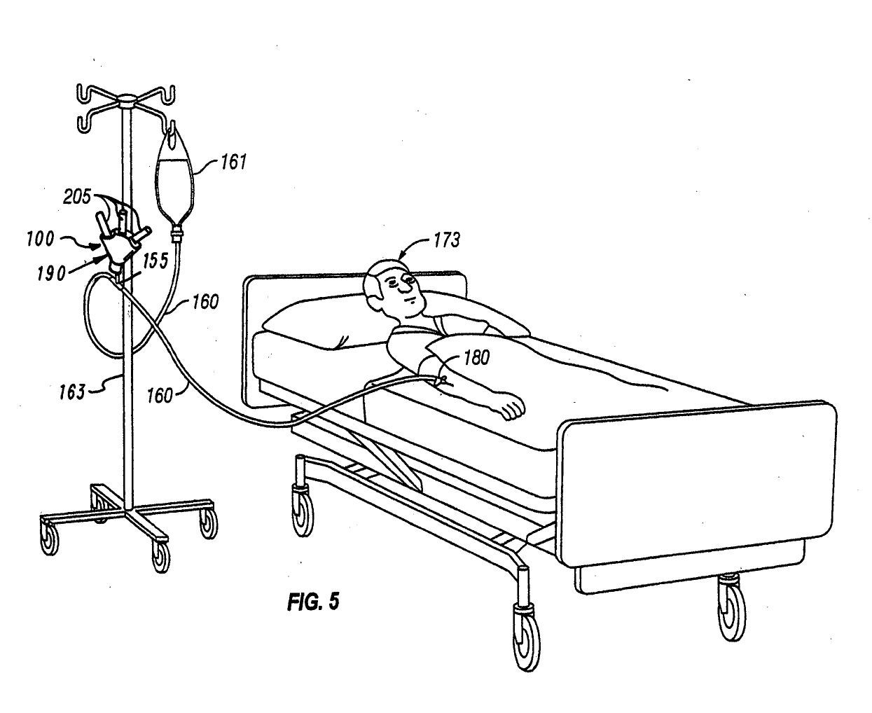

Infusion device 100 is particularly and advantageously adapted to be connected to a conventional wye port or injection site 155 operably connected to an intravenous conduit 160, FIGS. 1 and 5. Injection site 155 may comprise the so-called female side of a Luer connector or fitting, the male side comprising the fitting or connector part 150 including a conduit section 151 connected to manifold 140 by way of outlet conduit 140d. As shown in FIG. 5, connector 155 is secured to intravenous conduit or tubing 160 leading from an intravenous solution bag 161 at one end to a patient entry point on a patient 173, such as a catheter needle 180, on the other end. As mentioned previously, the connector 150 is preferably a male Luer type, which is advantageous in view of the convention for intravenous injection site connectors, such as element 155, being characterized as of the female Luer type. Moreover, the output conduit section 151 and connector part 150 of the device 100 advantageously replaces the conventional prior art sharp spike type devices used to pierce and join an incoming line to the intravenous line or tubing 160. The tubing connector or injection site structure 155 may also be integral with and oriented at an acute angle with respect to the intravenous tubing 160. Accordingly, the configuration of the infusion device of the present invention eliminates the need to create a break in the so-called sterile field of an intravenous fluid delivery system.

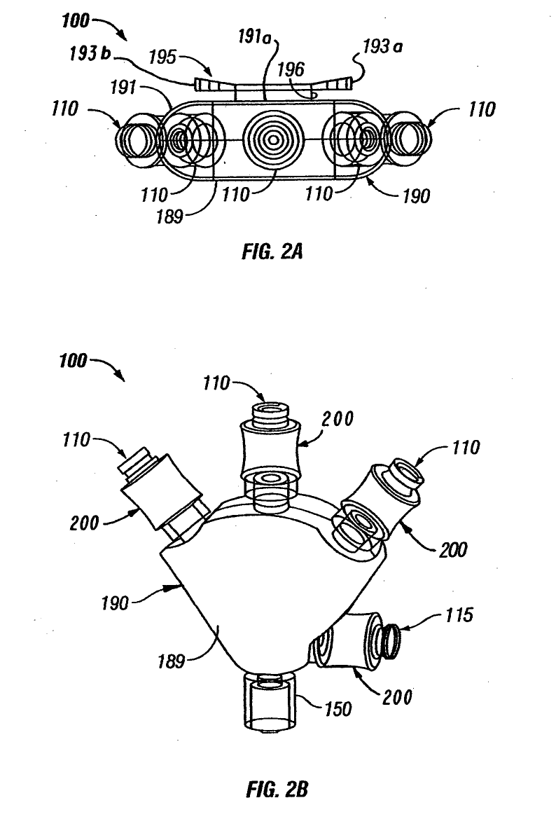

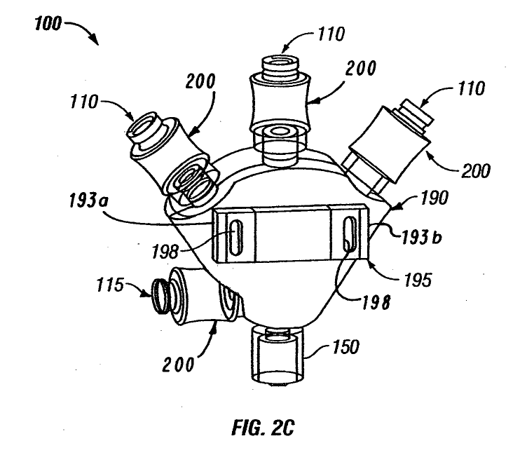

FIG. 2A is a top plan view of the infusion device 100 shown in FIG. 1, with both a front side 189 and an opposed back side 191 of a shell-like housing 190 in place. Housing 190 is preferably formed of molded plastic and is provided to facilitate protection for and handling of device 100 and to provide a support for a device mounting bracket 195. The back side 191 of the housing 190 includes mounting bracket 195 integrally formed thereon and which comprises a generally rectangular block-like support stub 196 supporting a pair of opposed somewhat wedge shaped wings 193a and 193b extending in opposite directions away from the support stub 196 and also standing off from a wall surface 191a of housing back side 191. FIG. 2B is a front perspective view of the infusion device 100 depicted in FIG. 1, with both the front and back sides 189 and 191 of the housing 190 in place. FIG. 2C is a perspective view of the infusion device 100 showing the mounting bracket 195 and the fourth port 115, with both the front and back sides 189 and 191 of the housing 190 in place but, as in FIG. 2B, the housing is not shown as transparent.

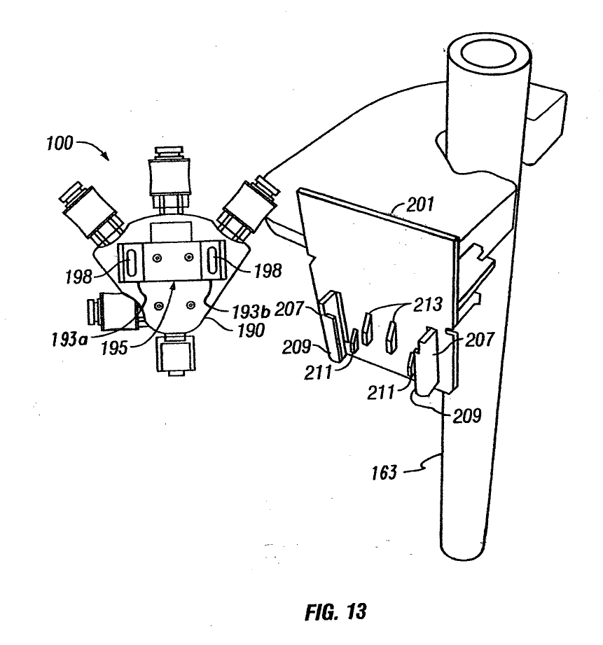

One or both of the wings 193a and 193b of the mounting bracket 195 may be somewhat elastically deflectable to facilitate mounting the device 100 on a receiving bracket. The wings 193a and 193b each have a slot 198, FIG. 2C, formed therein and operable to receive a detent or protrusion on a receiving bracket to be described in more detail in connection with FIGS. 13-15.

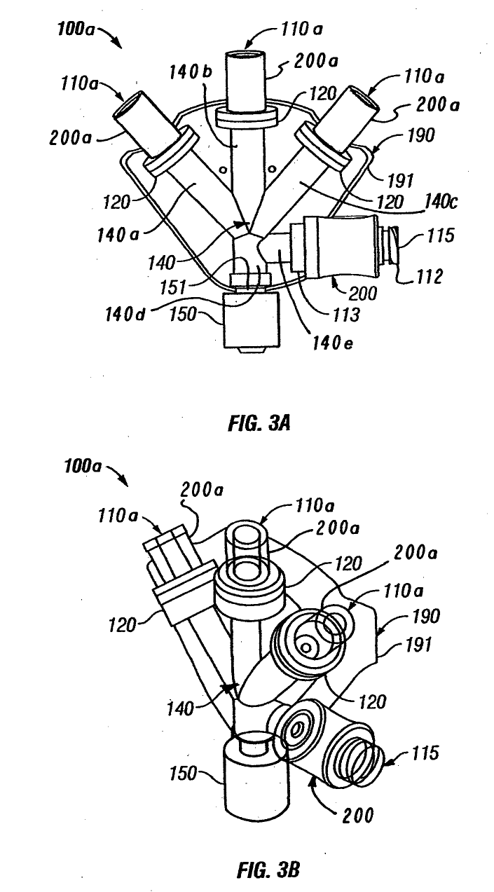

Referring now to FIGS. 3A and 3B, another embodiment of an infusion device 100a, depicts the front housing side 189 and the three upper self-sealing receptacles 200 removed. The infusion device 100a also includes check valves 120, the manifold 140, the fourth port 115 and a self-sealing swabable valve 200 mounted to Luer fitting 113 for connecting to a source of fluid, not shown, or for evacuating fluids, including trapped air from manifold 140. Output conduit 151 includes and comprises part of Luer fitting 150 for connecting to the injection site connector 155, which is connected to tubing 160 leading to a patient entry point. Ports 110a are characterized as relatively short cylindrical, rigid plastic conduit or tubing type receptacles 200a for connection to fluid supply devices, not shown, respectively.

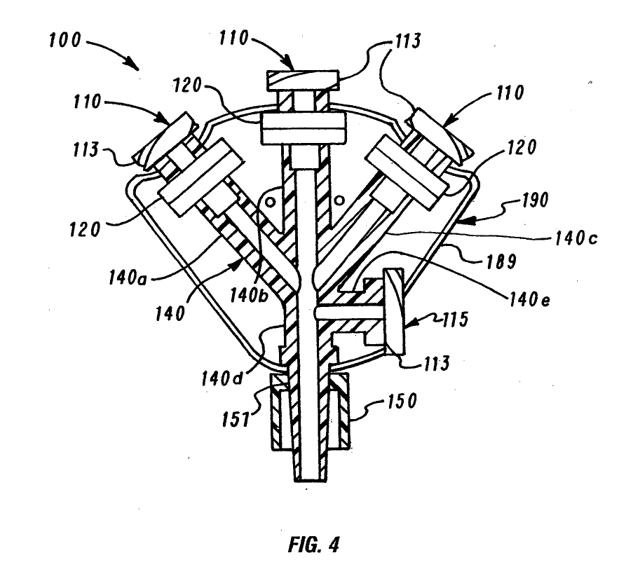

Referring now to FIG. 4, the infusion device 100 is shown in central section view with all self-sealing receptacles or swabable valves 200 removed. Thus, device 100 may be connected via Luer fittings 113, for example, to other fluid supply and fluid evacuation devices, respectively, if desired. FIG. 4 illustrates the internal passages of each of the conduits 140a, 140b, 140c, 140d, 140e and 151. These passages are all, preferably, of minimum diameter of about 1.0 mm, as indicated previously.

Referring now to FIG. 5, the perspective view shows a typical arrangement of the infusion device 100 connected to an intravenous line 160 that is in use to deliver medicine to an entry point on a patient 173, which is shown as a catheter needle 180 inserted into the patient's arm. Syringes 205 are shown connected to device 100 at respective ports 110 for infusion of suitable treatment fluids. Tube or line 160 is connected to fluid container 161 supported on transportable pole 163. Device 100 is mounted on pole 163 by way of an improved mounting arrangement to be described further herein.

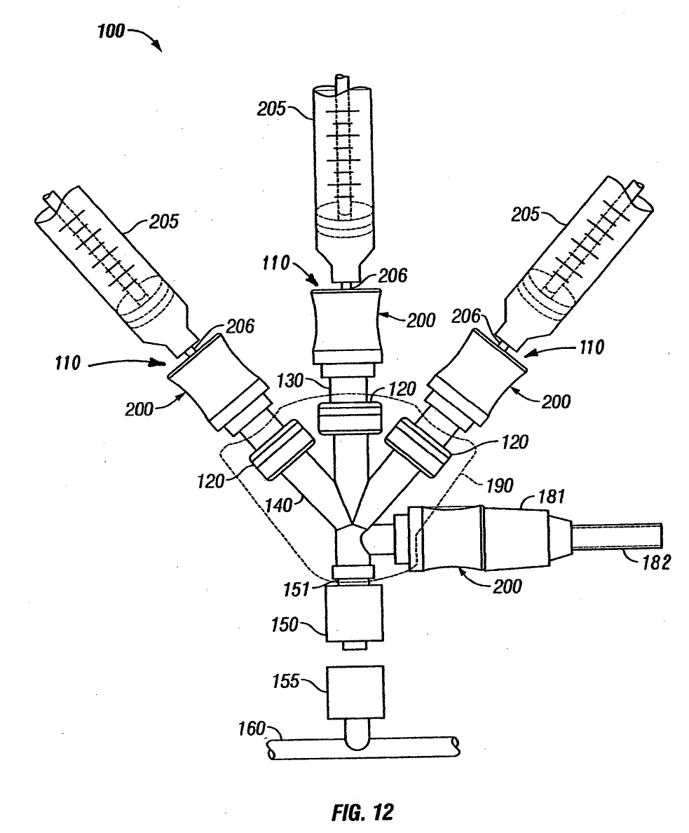

As shown in FIG. 12 also, for example, plural syringes 205 are aligned with the respective ports 110 of manifold 140 and are angled upward and away from each other by, preferably, about thirty degrees to forty-five degrees. Accordingly. the longitudinal central axes of conduits 140a, 140b and 140c extend at these same angles relative to each other, respectively. One of the advantages of manifold 140 with angled inlet ports 110 is that, as can be seen in FIG. 12, when syringes 205 are attached to the inlet self-sealing receptacles or valves 200, the syringes 205 will be spread apart somewhat so that there will remain a suitable clearance between them. This also facilitates injection of the fluid from the syringes, since the plunger thumb ends of each syringe 205 will be spread apart and easier to manipulate. In FIG. 12, the ports 110 are modified to not include Luer connector cam or thread elements 112. Accordingly, by orienting the conduit sections 140a, 140b and 140c as illustrated and described, easier manipulation of syringes or other fluid conducting structure leading to the device 100 or 100a is provided. The aforementioned angles between each of the conduit sections 140a, 140b, 140c and 140e with respect to conduit section 140d may be varied considerably. However, the range of angles described herein is preferable.

In use, a medical practitioner needing to infuse multiple fluids into the vascular system of a patient through a single patient entry point could proceed as follows. Using an antiseptic, the connector or injection site 155 would be swabbed to sterilize the connection point for the device 100 or 100a to the intravenous conduit or line 160. The sterile package containing the device 100 or 100a would be opened and the device removed. The device 100 or 100a, for example, would be connected to syringes or other sources of fluids to be injected to flush the respective inlet ports to remove air from the fluid passageways of the device. Entrapped air can also be removed from the manifold 140 by inverting and tapping the device. The device 100 or 100a would then be placed on a stable surface and a cap covering the outflow conduit section 151 would be removed. The device 100 or 100a would then be connected to the injection site or connector 155 after further clearing air from the passageways of the device. Thanks to the provision of the check valves 120, fluid flow will be unidirectional from the ports 110 when the receptacles or valves 200 are activated. If any air or other fluid to be evacuated remains in the passages of the manifold 140 such may be evacuated through the port 115 by connecting a syringe thereto or by connecting another suitable evacuation device to the port 115. Once the entire multiple agent infusion procedure is complete the device 100 may be disconnected from the injection site connector 155 and discarded per institutional guidelines.

As depicted in FIG. 11, fluid supply tubes 170 may be connected to one or more automatic metering pumps (not shown) and attached to self-sealing receptacles 200 and locked thereto by means of suitable Luer fittings 171 engaged with the Luer cams or threads 112. In such an arrangement the metering pump or pumps (not shown) may introduce a measured amount of each of the fluids through one or more of the tubes 170 into the respective inlet port or ports while the other tubes 170 remain installed on their respective ports, which fluid or fluids will ultimately be delivered to the patient through the tube 160. Because each of the inlet ports 110 is in fluid communication with a check valve 120, fluid from one of the tubes 170 will not flow backwards into another of the tubes 170.

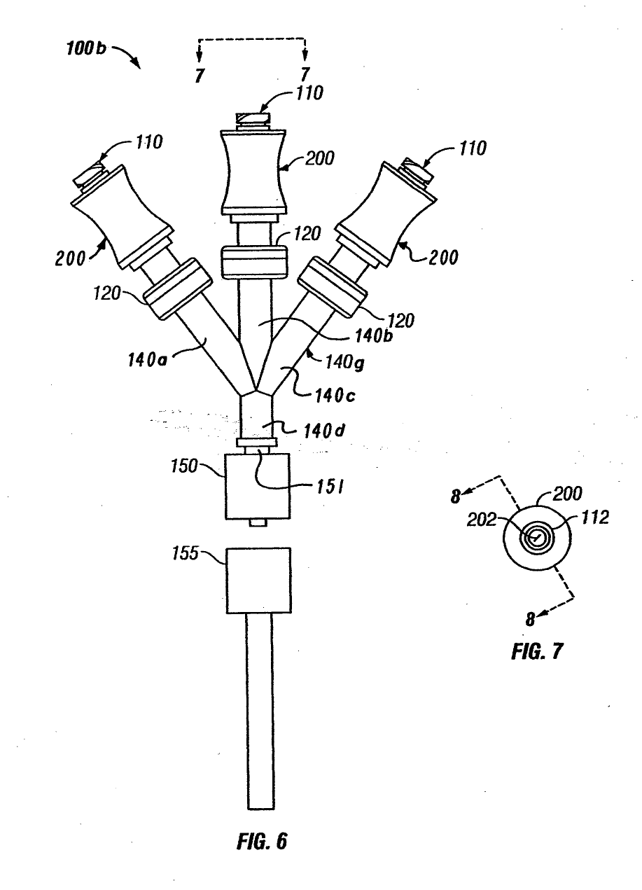

With reference now to FIG. 6, an embodiment of an infusion device 100b is shown having three inlet ports 110, each having a check valve 120 connected to a modified manifold 140g wherein the fourth inlet port 115 is omitted. Of course, the manifolds 140 or 140g may also include two inlet ports 110, or virtually any number of inlet ports 110. FIG. 7 is an end view of one of the self-sealing receptacles or valves 200 showing a self-sealing valve head 202 provided with a closable slit 202a shown in a valve closed position.

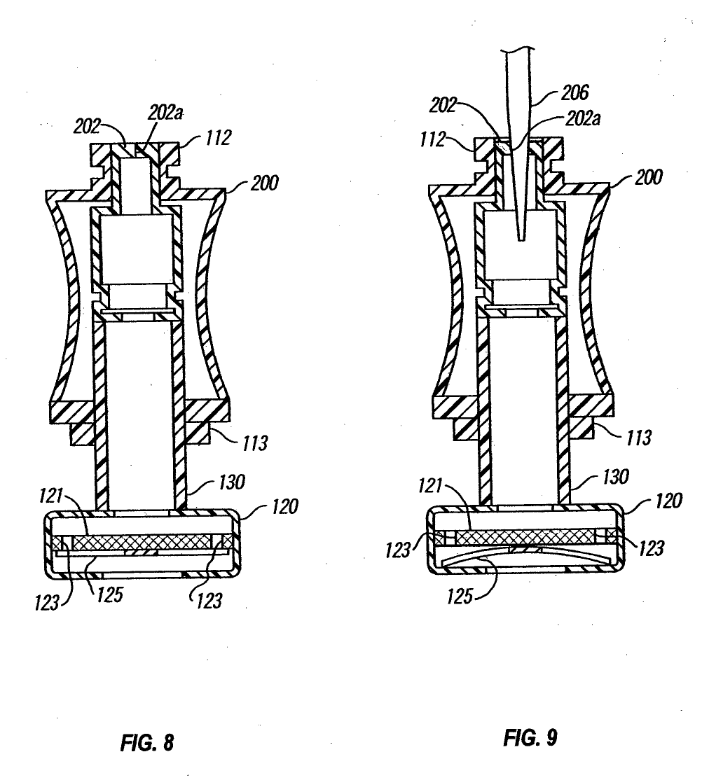

FIG. 8 is a cross-section taken through a self-sealing receptacle or valve 200 mounted to a Luer fitting 113 and illustrating one embodiment of the check valve 120 having a seat 121, ports 123 and a deflectable strip or disc type closure member 125. Swabable valves 200 each include a resilient deformable elastomer head 202 positioned within a substantially rigid hollow plastic housing, as shown, for access by swabbing the exposed outer surface of the head with a disinfectant in a known way. FIG. 9 is a cross-section taken through the self-sealing receptacle or valve 200 and the check valve 120, showing the valve 200 in a so-called open position, that is, with a syringe needle 206 inserted into and through slit 202a in valve head 202 and with valve closure member 125 in an open position.

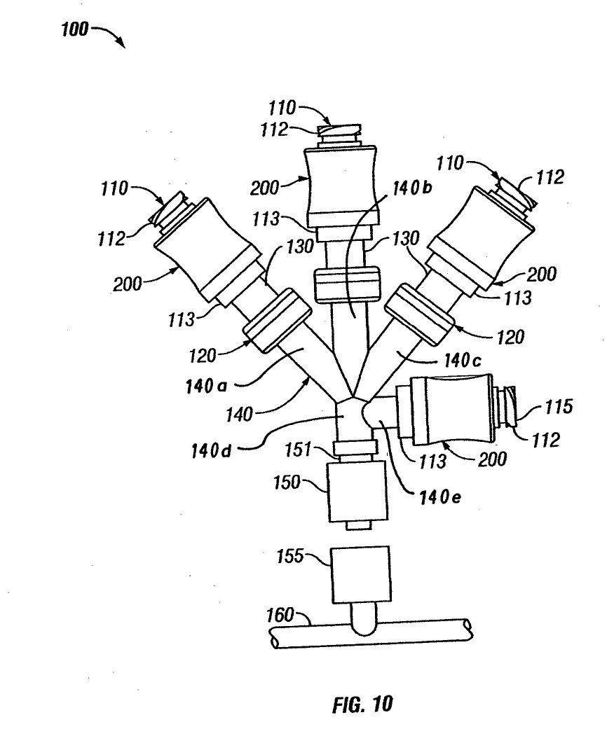

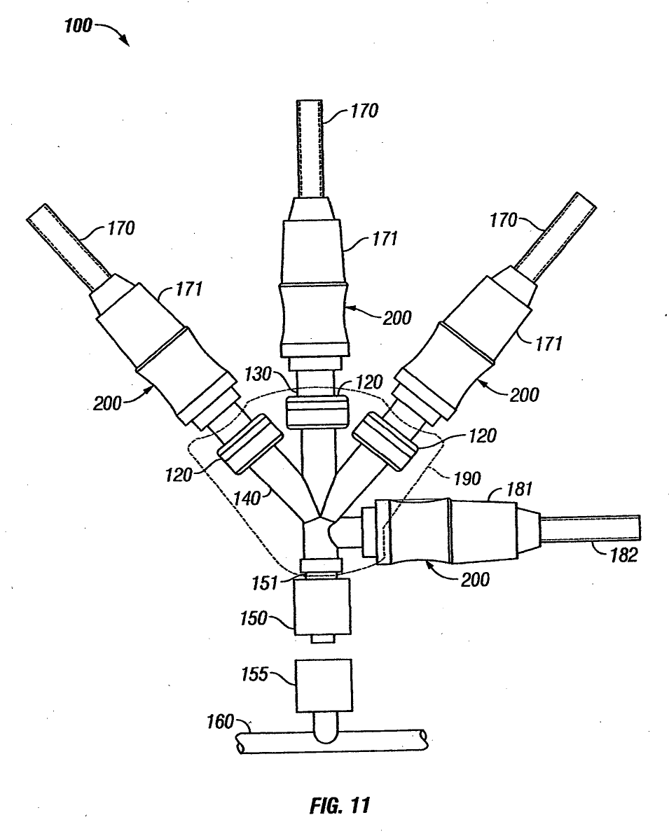

FIG. 10 also shows infusion device 100, with the front and back housing sides 189 and 191 removed and showing the three self-sealing receptacles 200 mounted to Luer fittings 113, check valves 120, manifold 140, the fourth port 115 and conduit 151 having a connector 150 thereon, all forming a substantially rigid structure. Apart from the convenience factor in handling devices 100 or 100a the housing 190 may not be required, but is somewhat advantageous as a support for bracket 195, also.

In FIG. 12, device 100 is shown with a syringe 205, having a needle 206, attached to each of the upper inlet ports 110 by forcing the needle through swabable valve head 202, as shown in FIG. 9. However, conventional needle receiving elastomer valves, not shown, may be provided in place of valves 200, if desired. The swabable valve 200 at port 115 may also be replaced by other suitable valve means. A tube 182 may be attached to the port 115 by a male Luer connector 181, for example, as shown.

During surgical procedures, there is often a considerable amount of movement of doctors, nurses and other health professionals around the patient. Such persons may accidentally come into contact with the infusion device including the syringes or the tubing connected to the device. Such action can possibly dislodge the syringes and/or tubing. To reduce the possibility of accidental dislodgement, the infusion device 100 includes the mounting bracket 195 which may be molded integrally with the manifold 140 or with the back side member 191 of the housing 190.

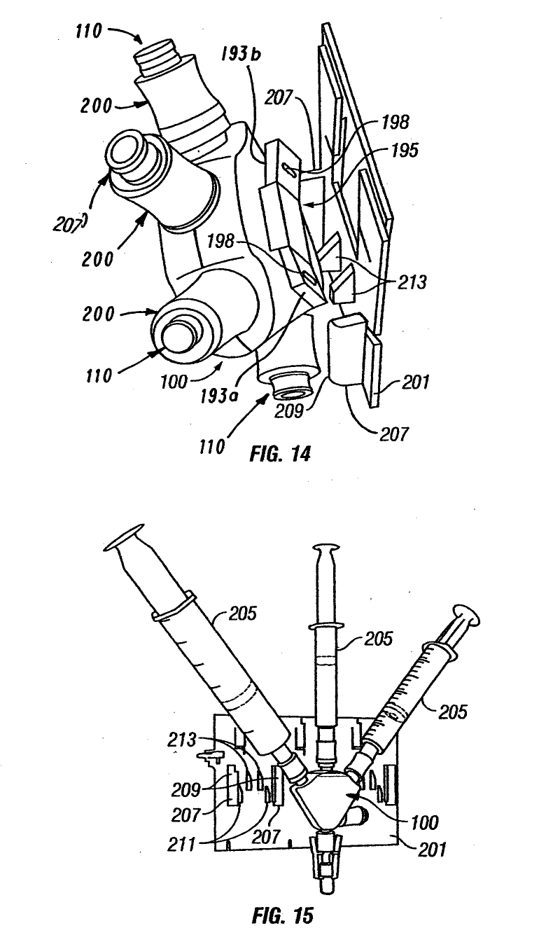

Referring now to FIGS. 13 and 14, the mounting bracket wings 193a and 193b slidably fit into a cooperating receiving bracket 201 having a pair of spaced apart parallel walls 207, each of which includes a retention lip or flange 209. The wings 193a and 193b are operable to be moved downwardly between the parallel walls 207 and underneath the retention lips 209 until at least one of the wings reaches a stop 211 in the receiving bracket 201. At the same position, projections 213 on the receiving bracket 201 protrude or snap into the slots 198 of the bracket 195 to secure the device 100. The infusion device 100 will then be retained in position on the receiving bracket 201 and less prone to being accidentally bumped in a way such that the syringes, tubings or other parts could be dislodged. Receiving bracket 201 is typical of hospital equipment support brackets and is mountable on pole 163, for example. FIG. 15 depicts the mounting bracket 195 installed onto another receiving bracket 201a. Receiving bracket 201a has multiple sets of support walls 207 and cooperating projections previously described.

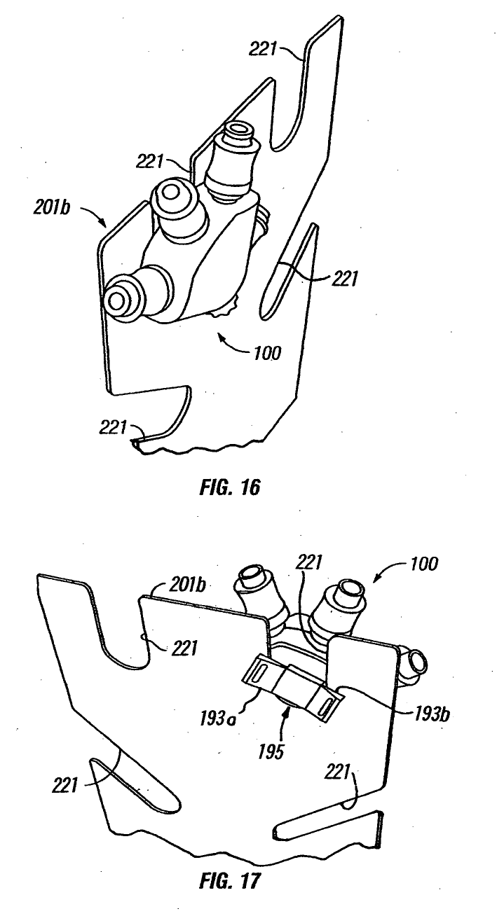

The bracket 195 is adaptable to being connected to different receiving brackets, another example of which is depicted in FIGS. 16 and 17. Flat-plate style receiving bracket 201b can be used, having one or more generally upwardly opening slots 221 formed therein. At least one upwardly open slot 221 is wider than the width of the bracket support stub 196 and narrower than the distance between opposite ends of the wings 193a and 193b. Thus, when the bracket 195 is inserted into a slot 221 of the bracket 201b, the device 100 will be maintained connected to the bracket as shown in FIG. 17.

Having described the present invention by reference to certain preferred embodiments, it is noted that the embodiments disclosed are illustrative rather than limiting in nature and that a range of variations, modifications, changes, and substitutions are contemplated and, in some instances, some features of the present invention may be employed without a corresponding use of the other features. It is appropriate that the appended claims be construed broadly and in a manner consistent with the scope of the invention.

1. A fluid infusion device for receiving fluids from multiple sources and for conducting fluids to an intravenous conduit, said infusion device comprising:
a manifold including plural inlet ports arranged in a pattern wherein each of said inlet ports is oriented at an angle with respect to at least one other of said inlet ports;
a manifold outlet conduit including a fitting disposed thereon for connection to a cooperating fitting leading to said intravenous conduit; and
connectors at each of said inlet ports for connecting said manifold to multiple sources of fluids.

2. The device set forth in claim 1, wherein:
each of said inlet ports is connected to an inlet conduit including a one-way valve allowing flow of fluid from said inlet ports toward said manifold outlet conduit only.

3. The device set forth in claim 2, including:
a further port connected to said manifold between said one-way valves and said manifold outlet conduit for introducing fluids to said manifold or evacuating fluids from said manifold, respectively.

4. The device set forth in claim 2, including:
self-sealing receptacles connected to said manifold inlet conduits, respectively, and operable upon connection to a fluid injection device, respectively, to allow flow of fluid through said manifold to said manifold outlet conduit.

5. The device set forth in claim 4, wherein:
said self-sealing receptacles comprise swabable valves.

6. The device set forth in claim 1, including:
a mounting bracket for mounting said device on a receiving bracket.

7. The device set forth in claim 6, wherein:
said mounting bracket comprises a support stub and opposed wings standing off a distance sufficient to provide for connecting said mounting bracket to said receiving bracket by way of one or more slots formed in said receiving bracket.

8. The device set forth in claim 6, wherein:
said mounting bracket comprises a support stub and opposed wings standing off such as to provide for inserting said wings between opposed flanges on said receiving bracket.

9. The device set forth in claim 8, wherein:
said wings include slots for receiving cooperating detent projections on said receiving bracket.

10. The device set forth in claim 6, wherein:
said mounting bracket is connected to a housing for said device, said housing being connected to and enclosing at least a portion of said manifold.

11. The device set forth in claim 1 wherein:
said fitting disposed on said manifold outlet conduit includes means for connection to said cooperating fitting leading to said intravenous conduit without disconnecting said intravenous conduit from a source of intravenous fluid.

12. The device set forth in claim 1 wherein:
each of said inlet ports is connected to an inlet conduit connected to said outlet conduit, said conduits being of a length and passage diameter as to minimize the volume of fluid contained within said device.

13. A fluid infusion device for receiving fluids from multiple sources and for conducting fluids to an intravenous conduit, said infusion device comprising:
a manifold including plural inlet ports arranged in a pattern wherein each of said inlet ports is oriented at an acute angle with respect to at least one other of said inlet ports;
a manifold outlet conduit including a fitting disposed thereon for connection to a cooperating fitting leading to said intravenous conduit;
each of said inlet ports is connected to an inlet conduit including a one-way valve allowing flow of fluid from said inlet ports toward said manifold outlet conduit only;
a further port connected to said manifold between said one-way valves and said manifold outlet conduit for introducing fluids to or evacuating fluids from said manifold, respectively; and
connectors at each of said ports for connecting said manifold to multiple fluid conduits.

14. The device set forth in claim 13, including:
self-sealing receptacles connected to said manifold inlet conduits, respectively, and operable upon connection to fluid injection devices, respectively, to allow flow of fluid through said manifold to said manifold outlet conduit.

15. The device set forth in claim 14, wherein:
said self-sealing receptacles comprise swabable valves.

16. A fluid infusion device for receiving fluids from multiple sources and for conducting fluids to an intravenous conduit, said infusion device comprising:
a manifold including plural inlet ports arranged in a pattern wherein each of said inlet ports is oriented at an acute angle with respect to at least one other of said inlet ports;
a manifold outlet conduit including a fitting disposed thereon for connection to a cooperating fitting leading to said intravenous conduit;
connectors at each of said inlet ports for connecting said manifold to multiple sources of fluids; and
a mounting bracket for connecting said device to a receiving bracket of a hospital structure.

17. The device set forth in claim 16, wherein:
said mounting bracket comprises a support stub and opposed wings standing off a distance sufficient to provide for connecting said mounting bracket to said receiving bracket by way of one or more slots formed in said receiving bracket.

18. The device set forth in claim 16, wherein:
said mounting bracket comprises a support stub and opposed wings standing off such as to provide for inserting said wings between opposed flanges on said receiving bracket.

19. The device set forth in claim 18, wherein:
said wings include slots for receiving cooperating detent projections on said receiving bracket.

20. The device set forth in claim 16, wherein:
said mounting bracket is connected to a housing for said device, said housing being connected to and enclosing at least a portion of said manifold.

21. A fluid infusion device for receiving fluids from multiple sources and for conducting fluids to an intravenous conduit, said infusion device comprising:
a manifold including three inlet ports arranged in a pattern wherein each of said inlet ports is oriented at an acute angle with respect to at least one other of said inlet ports;
a manifold outlet conduit including a fitting disposed thereon for connection to a cooperating fitting leading to said intravenous conduit;
each of said inlet ports is connected to an inlet conduit including a one-way valve allowing flow of fluid from said inlet ports toward said manifold outlet conduit only;
a further port connected to said manifold between said one-way valves and said manifold outlet conduit for evacuating fluids from said manifold;
self-sealing receptacles connected to said manifold inlet conduits, respectively, and operable upon connection to a fluid injection device, respectively, to allow flow of fluid through said manifold to said manifold outlet conduit;
connectors at each of said inlet ports for connecting said manifold to said fluid injection devices; and
a mounting bracket for connecting said device to a device receiving bracket.

22. The device set forth in claim 21, wherein:
said self-sealing receptacles comprise swabable valves.

23. The device set forth in claim 21, wherein:
said mounting bracket comprises a support stub and opposed wings standing off a distance sufficient to provide for connecting said mounting bracket to said receiving bracket by one of slots formed in said receiving bracket and opposed flanges on said receiving bracket.

24. A fluid infusion device for receiving fluids from multiple sources and for conducting fluids to an intravenous conduit, said infusion device comprising:
a manifold including at least three inlet ports arranged in a pattern wherein each of said inlet ports is oriented at an acute angle with respect to at least one other of said inlet ports;
each of said inlet ports being rigidly connected to a rigid inlet conduit section interconnected with a rigid manifold outlet conduit including a fitting disposed thereon for connection to a cooperating fitting leading to said intravenous conduit;
each of said inlet conduit sections being connected to a one-way valve allowing flow of fluid from said inlet ports toward said manifold outlet conduit only;
a further port connected to said manifold by a rigid conduit section between said one-way valves and said manifold outlet conduit for evacuating fluids from said manifold;
each of said inlet ports including receptacles connected to said manifold inlet conduit sections, respectively, and operable upon connection to a fluid injection device, respectively, to allow flow of fluid through said one way valves and said manifold to said manifold outlet conduit;
a rigid two part housing disposed on and enclosing at least a portion of said manifold; and
a mounting bracket formed integral with one part of said housing for connecting said device to a device receiving bracket.

25. The device set forth in claim 24 including:
connectors at each of said inlet ports for connecting said manifold to said fluid injection devices.

26. The device set forth in claim 24, wherein:
said mounting bracket comprises a support stub and opposed wings standing off a distance sufficient to provide for connecting said mounting bracket to said receiving bracket by one of a cooperating slot formed in said receiving bracket and opposed flanges on said receiving bracket.

27. The device set forth in claim 24, wherein:
said receptacles comprise self-sealing swabable valves.

28. A fluid infusion device for receiving fluids from multiple sources and for conducting fluids to an intravenous conduit, said infusion device comprising:
a manifold including plural inlet ports arranged in a pattern wherein each of said inlet ports is oriented at an acute angle with respect to another of said inlet ports;
each of said inlet ports being rigidly connected to a rigid inlet conduit section interconnected with a rigid manifold outlet conduit, said outlet conduit including a fitting disposed thereon for connection to a cooperating fitting leading to said intravenous conduit, said fitting including means for connection to said cooperating fitting without disconnecting said intravenous conduit from a source of intravenous fluid;
each of said inlet conduit sections being connected to a one-way valve allowing flow of fluid from said inlet ports toward said manifold outlet conduit only; and
each of said inlet ports including receptacles connected to said manifold inlet conduit sections, respectively, and operable upon connection to a fluid injection device, respectively, to allow flow of fluid through said one way valves and said manifold to said manifold outlet conduit.
